(19) United States
(12) Patent Application Publication
(10) Pub. No.: US 2008/0119717 A1
(43) Pub. Date: May 22, 2008

(54) INTERACTIVE PROTOCOLING BETWEEN A RADIOLOGY INFORMATION SYSTEM AND A DIAGNOSTIC SYSTEM/MODALITY

(75) Inventors: Mark Vincent Profio, Elm Grove, WI (US); Kimberly Rae Stavrinakis, Richmond, VT (US); Sang Lee, Stevensville, MI (US); Regan Park Fields, Berea, OH (US); Christopher James Proctor, Aurora, CO (US); Holly Ann McDaniel, Waukesha, WI (US); David Alan Littlejohn, Wales, WI (US); Tony Edward Palmer, Williston, VT (US)

Correspondence Address: MCANDREWS HELD & MALLOY, LTD, 500 WEST MADISON STREET, SUITE 3400, CHICAGO, IL, 60661, US

(73) Assignee: GENERAL ELECTRIC COMPANY, Schenectady, NY (US)

(21) Appl. No.: 11/857,980

(22) Filed: Sep. 19, 2007

Related U.S. Application Data
(60) Provisional application No. 60/867,007, filed on Nov. 22, 2006.

(51) Int. Cl. A61B 5/00

(52) U.S. Cl. 600/407

(57) ABSTRACT

Certain embodiments of the present invention provide methods and systems for interactive protocoling between a healthcare information system and a diagnostic imaging system/modality. Certain embodiments provide a method for interactive protocoling between healthcare information and diagnostic imaging systems including viewing available scanning protocols for a diagnostic imaging system and associating a patient scanning order with one or more of the available scanning protocols for the diagnostic imaging system. Certain embodiments provide an interface enabling interactive protocoling between healthcare information and diagnostic imaging systems. The interface includes a protocol list including available scanning protocols for a diagnostic imaging system and a dialog for accepting input from a user to specify one or more of the available scanning protocols from the protocol list. The dialog is configured to associate an order for scanning a patient using the diagnostic imaging system with the one or more specified scanning protocols.

### RELATED APPLICATIONS

This application claims priority to provisional application Ser. No. 60/867,007, filed on Nov. 22, 2006, entitled “Interactive Protocoling Between a Radiology Information System and a Diagnostic System/Modality,” which is herein incorporated by reference in its entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to communication between a healthcare information system and an imaging system or modality. In particular, the present invention relates to interactive protocoling between a healthcare information system and a diagnostic imaging system/modality.

A scanning protocol defines one or more imaging scans and associated actions to be performed on a patient. For example, a head CT scanning protocol may include a specification of table speed, gantry rotation rate, pitch, collimation, focal point, contrast, etc. Scanning protocols may vary depending upon Diagnostic Imaging (DI) scanner type and software used, for example.

Today's healthcare information systems, such as Radiology Information Systems (RIS), Picture Archiving and Communication Systems (PACS), etc., have limited or no access to detailed protocol capabilities on specific Diagnostic Imaging scanner systems. The exercise of “protocoling”, or matching a referring physicians patient order with a specific procedure and scanning protocol on a Diagnostic Imaging modality (e.g., X-ray, computed tomography (CT), digital radiography (DR), magnetic resonance (MR), positron emission tomography (PET), ultrasound, etc.) prior to the procedure, is today a general association and may not exploit the particular strengths of specific DI systems.

### BRIEF SUMMARY OF THE INVENTION

Certain embodiments of the present invention provide methods and systems for interactive protocoling between a healthcare information system and a diagnostic imaging system/modality.

Certain embodiments provide a method for interactive protocoling between a healthcare information system and a diagnostic imaging system including viewing available scanning protocols for a diagnostic imaging system and associating a patient scanning order with one or more of the available scanning protocols for the diagnostic imaging system.

Certain embodiments provide an interface enabling interactive protocoling between a healthcare information system and a diagnostic imaging system. The interface includes a protocol list including available scanning protocols for a diagnostic imaging system and a dialog for accepting input from a user to specify one or more of the available scanning protocols from the protocol list. The dialog is configured to associate an order for scanning a patient using the diagnostic imaging system with the one or more specified scanning protocols.

Certain embodiments may be implemented in a computer readable medium having a set of instructions for execution by a computer, for example. The computer-readable medium and its instructions may be used to provide interactive protocoling as described above. For example, certain embodiments may be implemented as a set of instructions including a protocol library, a protocol selection routine, and an order routine. The protocol library includes available scanning protocols for a diagnostic imaging system, for example. The protocol selection routine displays the available scanning protocols and accepts input from a user to specify one or more of the available scanning protocols from the protocol library. The protocol selection routine is configured to associate an order for scanning a patient using the diagnostic imaging system with the one or more specified scanning protocols. The order routine updates the order information to reference the associated protocol(s) for the diagnostic imaging system. The diagnostic imaging system may use functionality, such as a DICOM modality worklist (MWL) functionality, to retrieve its pending orders, for example.

### BRIEF DESCRIPTION OF SEVERAL VIEWS OF THE DRAWINGS

FIG. 1 illustrates a system and flow diagram for interactive protocoling in accordance with an embodiment of the present invention.

FIG. 2 shows an exemplary interface for a user at a healthcare information system and/or workstation in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

A scanning protocol specifies a combination of modality settings and/or methods for communication and/or operation, for example. Interactive protocoling provides a communication link between diagnostic imaging (DI) systems and a healthcare information system, such as a RIS (which will be discussed herein for purposes of illustration only). With interactive protocoling, the RIS may “look into” specific scanning protocols available on a target DI system, allowing the specific association of a patient procedure/order with an optimal DI system protocol for that patient case.

Scanning protocol(s) may be automatically selected based on one or more criteria and/or manually selected from a provided list given certain constraints, for example. A scanning protocol may identify a sequence of views to be obtained, for example. For example, a “standard chest X-ray” protocol may specify anteroposterior and lateral views. A procedure may also be specified by a scanning protocol, including and/or referring to multiple sub- or constituent protocols, for example. For example, a scanning protocol may specify actions to perform a particular procedure or procedure step. In certain embodiments, a scanning protocol is specified by a code (e.g., a numeric or alphanumeric code). In certain embodiments, a user and/or system, such as a DI system, may process the components of a scanning protocol. For example, a modality may execute the steps (e.g., views or scans) specified in a selected or prescribed scanning protocol. In certain embodiments, a system and/or user may accept a “standard” or preset scanning protocol. In other embodiments, a system and/or user may modify a standard or preset scanning protocol according to one or more rules and/or preferences, for example.

Interactive Protocoling utilizes an IHE (Integrating the HealthCare Enterprise) protocol to define bi-directional communication between a RIS and one or more DI systems. IHE, for example, may provide one or more libraries and/or guidelines for communication and/or scanning protocols between and/or for clinical systems. Interactive Protocoling allows RIS users to view the scanning protocols on a specific DI system. The RIS user can then better specify a patient radiology order with 1) an appropriate DI system and 2) a specific scanning protocol on that system.

For example, some DI systems may be more appropriately chosen for Cardiac imaging or Neuro imaging. Bi-directional communication provided by Interactive Protocoling allows users to better match patients and cases to DI systems. Additionally, within a DI system, different scanning protocols may be better suited to certain patients based on a variety of factors including patient size, patient blood test results, and patient indications for allergies and medications, for example. Bi-directional communication provided by Interactive Protocoling allows users to better match patients with tailored scanning protocols.

IHE is an initiative by healthcare professionals and industry to improve the way computer systems in healthcare share information. IHE promotes the coordinated use of established standards such as DICOM and HL7 to address specific clinical needs in support of optimal patient care.

Patient care may be improved through efficient access to comprehensive electronic health records (EHRs). IHE accelerates the adoption of the information standards needed to support EHRs. IHE helps improve patient care by harmonizing healthcare information exchange and provides a common standards-based framework for seamlessly passing health information among care providers, enabling local, regional and national health information networks.

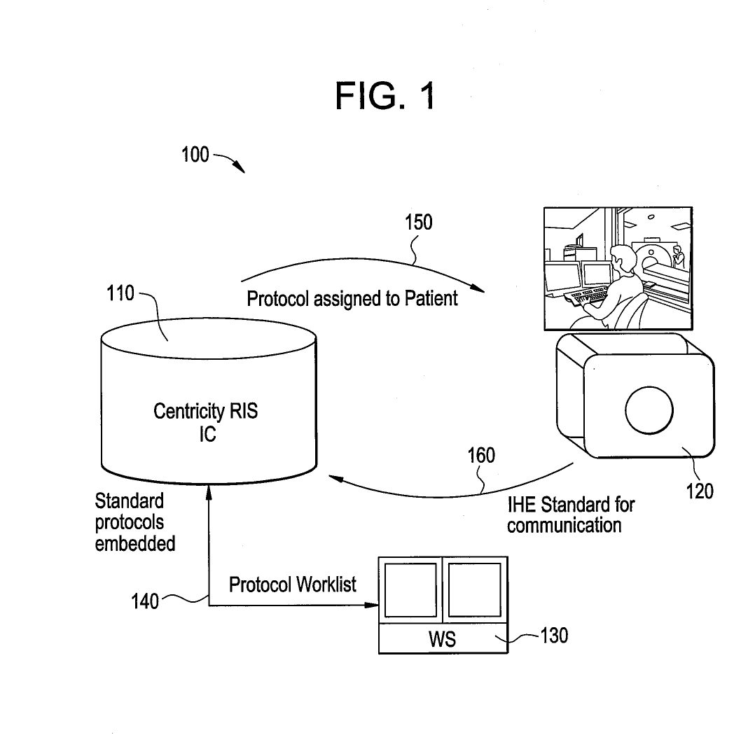

FIG. 1 illustrates a block and flow diagram for a system 100 for interactive protocoling in accordance with an embodiment of the present invention. The system includes a RIS 110, a DI system 120, and a workstation 130. In certain embodiments, standard scanning protocols may be embedded/stored at the RIS 110 and/or DI system 120 for user and/or automated review. User-defined scanning protocols may also be uploaded to the RIS 110 and/or DI system 120 from the workstation 130 and/or other source, for example. Scanning protocols viewable from the RIS 110 and/or workstation 130 may include protocols available on and/or for a particular DI system 120, for example.

At 140, a protocol worklist/workflow may be used to associate a scanning protocol with a patient order for a DI system 120. For example, a user may review a particular order (e.g., a patient procedure order) from the RIS 110 and/or workstation 130. The user selects an interactive protocoling option via the RIS 110 (and/or workstation 130 associated with the RIS 110). The RIS 110, for example, retrieves a list of DI systems 120 and presents the list to the RIS user. The user selects the DI system to be associated with the order. The RIS 110 retrieves a list of scanning protocols for the selected DI system 120 and presents the list to the user. The user selects the appropriate scanning protocol(s) for the DI system 120 and order.

At 150, the user may associate a patient radiology order for the DI system 120 with the selected scanning protocol(s) for that system 120. For example, a user at the RIS 110 can review the scanning protocols on the DI system 120 and associate a patient radiology order with an appropriate DI system 120 and a specified scanning protocol on that system 120.

The DI system 120 receives a list of its orders (e.g., via DICOM MWL). A DI system 120 user may select an order for the next patient who has an associated scanning protocol. The user executes the scanning protocol(s) identified in the order. The DI system 120 uses DICOM MPPS, for example, to communicate details of the procedure performed.

At 160, scanning information, patient information and/or a protocol summary may be sent back to the RIS 110 at completion of image acquisition at the DI system/modality 120. Such information may be stored and/or analyzed by the user, for example. In certain embodiments, information is communicated from the DI system 120 to the RIS 110 using an IHE-acceptable standard for communication, for example.

In certain embodiments, if a patient order is a repeat procedure, the RIS 110 determines that the patient has had this procedure before and retrieves the prior study information. The user specifies whether or not to follow the same procedure plan on the same DI system 120 as in the prior study.

In certain embodiments, for example, interactive protocoling utilizes an IHE Radiology Technical Framework—Scheduled Workflow Profile. The scheduled workflow profile relates to expected interaction between a scanner (e.g., a DI system) and a RIS with respect to scanning protocols. The IHE scheduled workflow profile includes an “Assisted Acquisition Protocol Setting Option” which outlines how to automate mapping a RIS protocol to a scanner protocol. Additional information regarding the IHE Radiology Technical Framework can be found at http://www.ihe.net/Technical_Framework/index.cfm#radiology, which is herein incorporated by reference in its entirety.

Current IHE profiles do not couple the RIS into the scanner protocols. Instead, IHE profiles, such as the scheduled workflow profile, outline a mapping between exam order-to-RIS protocol codes and, on the DI scanner, a mapping from RIS protocol code to scanner protocol. Certain embodiments facilitate a workflow and user interface to retrieve a list of scanner protocols from a particular scanner, associate a patient procedure order with a scanner protocol, and pass the selected scanner protocol to the scanner with the patient procedure order information, for example.

Certain embodiments extend a scheduled workflow and/or profile definition to allow such selection of a scanner protocol, association with a patient procedure, and transmission to a DI scanner. A scheduled workflow profile allows association of an order with a specific DI system (scanner). The SWF profile and DICOM MPPS (Modality Performed Procedure Step) outline what information the scanner returns to the RIS at completion of the image acquisition, for example. A DICOM modality worklist (MWL) defines a mechanism by which the scanner can retrieve a list of orders assigned to the scanner.

One or more of the steps of the flow diagram for the system 100 may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

In certain embodiments, for example, Interactive Protocoling helps enable a paperless approach to scheduling patients for DI system procedures. Referring physicians may access a Healthcare institution/facilities RIS system over the Internet or network. The physician can enter a report asking a Radiology department to provide relevant imaging to aid in patient condition diagnosis. Utilizing the Interactive Protocoling capability, a Radiologist can then scan the suite of available DI systems and make the most appropriate match between the patient need and DI system capabilities. Similarly, with the patient history available via the RIS, the Radiologist may then also specify the most appropriate scanning protocol for the patient.

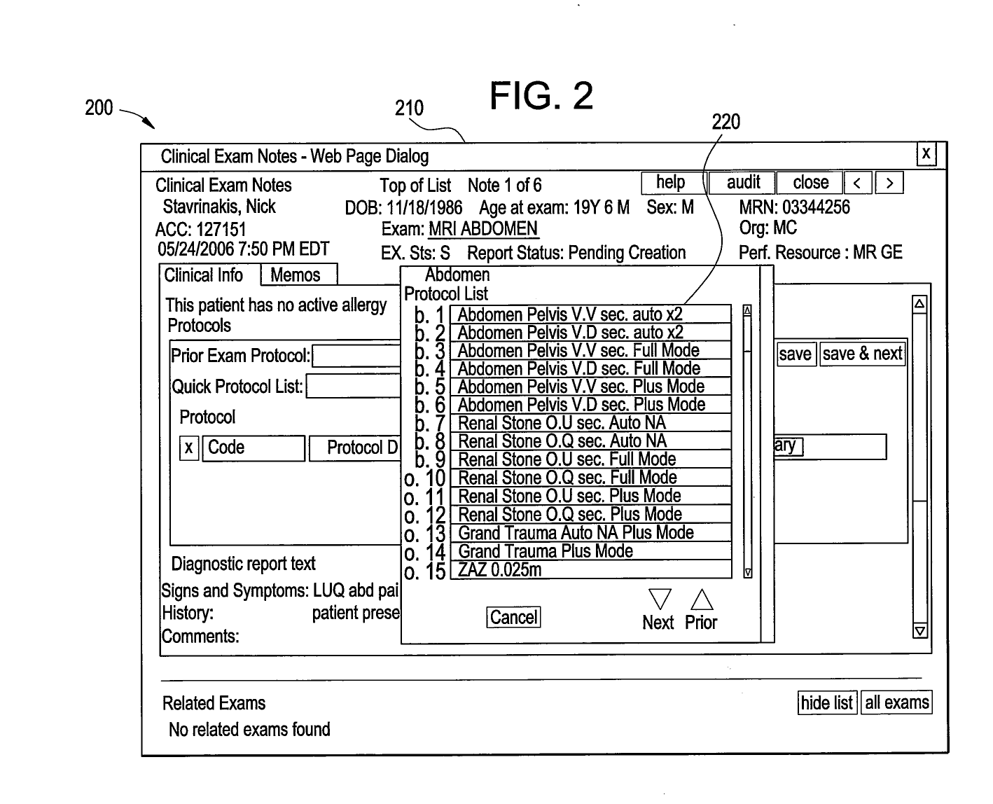

FIG. 2 shows an exemplary interface 200 for a user at a healthcare information system, such as a RIS, and/or workstation in accordance with an embodiment of the present invention. The interface 200 includes a dialog 210, such as a web page dialog box or window for review and/or entry of clinical exam notes on a RIS 110 and/or workstation 130, for example. The dialog 210 includes information such as patient information, exam or other order information, protocol information and report information.

A user may view a list of available scanning protocols via the dialog 210. An exemplary list is shown as protocol list 220 in FIG. 2. The protocol list 220 may be specific to an imaging system/modality, anatomy and/or procedure, for example. As shown in FIG. 2, the exemplary protocol list 220 lists available scanning protocols for an abdominal scan. Protocols may specify anatomy/area with an anatomy, timing, mode, etc. Protocols may specify a type of imaging or scanning, a view, etc. A user may select one or more protocols for use with respect to a patient from the list 220 via the dialog 210. In certain embodiments, one or more protocols may be default options and/or automatically selected based on one or more criteria including exam, equipment, resource availability, etc.

The selected protocol information may then be transmitted to the imaging system/modality (and/or a technician operating the imaging system/modality) for use in scanning the patient. The protocol information may be used to manually drive a technician operating the system/modality and/or to automatically drive the system/modality according to the steps and/or other elements included in the protocol, for example. The protocol information may be transmitted with an associated patient order, for example.

Thus, certain embodiments provide better patient care through interactive protocol. Interactive protocol provides more reliable and repeatable results for follow-up patient diagnostic imaging studies. Additionally, interactive protocoling helps provide improved accuracy in patient reports. In certain embodiments, interactive protocoling provides a technical effect of providing a listing or view of available scanning protocols at a DI system and allowing selection of one or more available protocols via a healthcare information system.

Interactive Protocoling provides time, efficiency, and quality control benefits to providers of Diagnostic Imaging services. Better patient care, more reliable and repeatable results for follow up patient diagnostic imaging studies, and improved accuracy in patient reports can all be better achieved with interactive protocoling.

Certain embodiments may be implemented in a computer readable medium having a set of instructions for execution by a computer, for example. The computer-readable medium and its instructions may be used to provide interactive protocoling as described above. For example, certain embodiments may be implemented as a set of instructions including a protocol library, a protocol selection routine, and an order routine. The protocol library includes available scanning protocols for a diagnostic imaging system, for example. The protocol selection routine displays the available scanning protocols and accepts input from a user to specify one or more of the available scanning protocols from the protocol library. The protocol selection routine is configured to generate an order for scanning a patient using the diagnostic imaging system with the one or more specified scanning protocols. The order routine transmits the generated order to the diagnostic imaging system. In certain embodiments, the protocol selection routine facilitates entry of examination notes from the diagnostic imaging system. In certain embodiments, the protocol library, the protocol selection routine, and the order routine may be executed at a healthcare information system and/or a workstation, for example.

The components, elements, and/or functionality of the interface(s) and system(s) described above may be implemented alone or in combination in various forms in hardware, firmware, and/or as a set of instructions in software, for example. Certain embodiments may be provided as a set of instructions residing on a computer-readable medium, such as a memory or hard disk, for execution on a general purpose computer or other processing device, such as, for example, a PACS workstation or one or more dedicated processors.

Several embodiments are described above with reference to drawings. These drawings illustrate certain details of specific embodiments that implement the systems and methods and programs of the present invention. However, describing the invention with drawings should not be construed as imposing on the invention any limitations associated with features shown in the drawings. The present invention contemplates methods, systems and program products on any machine-readable media for accomplishing its operations. As noted above, the embodiments of the present invention may be implemented using an existing computer processor, or by a special purpose computer processor incorporated for this or another purpose or by a hardwired system.

As noted above, certain embodiments within the scope of the present invention include program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Certain embodiments of the invention are described in the general context of method steps which may be implemented in one embodiment by a program product including machine-executable instructions, such as program code, for example in the form of program modules executed by machines in networked environments. Generally, program modules include routines, programs, objects, components, data structures, etc., that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Certain embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for interactive protocoling between a healthcare information system and a diagnostic imaging system, said method comprising:
viewing available scanning protocols for a diagnostic imaging system via a healthcare information system; and
associating a patient order with one or more of the available scanning protocols for the diagnostic imaging system via a control at the healthcare information system.

2. The method of claim 1, wherein the patient order relates to a patient procedure.

3. The method of claim 2, further comprising associating a patient procedure with a diagnostic imaging system protocol for execution with respect to a patient at the diagnostic imaging system.

4. The method of claim 1, further comprising interacting with the one or more available scanning protocols in the specified patient scanning order via a dialog at the healthcare information system.

5. The method of claim 1, further comprising identifying a prior patient order related to the patient order and retrieving scanning protocol information for the prior patient order for use with the patient order via the healthcare information system.

6. The method of claim 1, wherein the healthcare information system comprises at least one of a radiology information system and a picture archiving and communications system.

7. An interface enabling interactive protocoling between a healthcare information system and a diagnostic imaging system, said interface accessible from the healthcare information system, said interface comprising:
a protocol list including available scanning protocols for a diagnostic imaging system; and
a dialog for accepting input from a user at the healthcare information system to specify one or more of the available scanning protocols from the protocol list, the dialog configured to associate an order for scanning a patient using the diagnostic imaging system with the one or more specified scanning protocols.

8. The interface of claim 7, wherein the protocol list and the dialog are provided to a user via the healthcare information system.

9. The interface of claim 7, wherein the protocol list and the dialog are provided to a user via a workstation.

10. The interface of claim 7, wherein the patient scanning order relates to a patient procedure.

11. The interface of claim 10, wherein the patient procedure is associated with a diagnostic imaging system protocol for execution with respect to a patient at the diagnostic imaging system.

12. The interface of claim 7, wherein a user modifies the one or more available scanning protocols in the order via the dialog.

13. The interface of claim 7, wherein the dialog facilitates identification of a prior patient order related to the order for scanning the patient and retrieval of scanning protocol information for the prior patient order for use with the order for scanning the patient.

14. The interface of claim 7, wherein the healthcare information system comprises at least one of a radiology information system and a picture archiving and communications system.

15. The interface of claim 7, wherein available scanning protocols comprise at least one of an anatomy to be scanned, a view, timing, and a scanning mode.

16. The interface of claim 7, wherein the dialog facilitates entry of examination notes from the diagnostic imaging system.

17. A computer-readable medium having a set of instructions for execution on a computer, the set of instructions comprising:
a protocol library including available scanning protocols for a diagnostic imaging system;
a protocol selection routine for displaying the available scanning protocols and accepting input from a user to specify one or more of the available scanning protocols from the protocol library, the protocol selection routine configured to associate an order for scanning a patient using the diagnostic imaging system with the one or more specified scanning protocols; and
an order routine for updating the order to reference the associated one or more specified scanning protocols for the diagnostic imaging system.

18. The computer-readable medium of claim 17, wherein available scanning protocols comprise at least one of an anatomy to be scanned, a view, timing, and a scanning mode.

19. The computer-readable medium of claim 17, wherein the protocol selection routine facilitates entry of examination notes from the diagnostic imaging system.

20. The computer-readable medium of claim 17, wherein the protocol library, the protocol selection routine, and the order routine may be executed at one or more of a healthcare information system and a workstation.
